SEQUENCE LISTING (16 sequences)

SEQ ID NO: 1 - DNA, 18 bp, Artificial
  synthetic
accggatggccaatccaa

SEQ ID NO: 2 - DNA, 16 bp, Artificial
  synthetic
cctccgcccctgaatg

SEQ ID NO: 3 - DNA, 23 bp, Artificial
  synthetic
tctttaacgcgtcgaccttttat

SEQ ID NO: 4 - DNA, 21 bp, Artificial
  synthetic
aaatacgcgtctatcggttcc

SEQ ID NO: 5 - DNA, 27 bp, Artificial
  synthetic
gttagagtgaatgtttatctttcagca

SEQ ID NO: 6 - DNA, 22 bp, Artificial
  synthetic
ggttgttataccaacttgggga

SEQ ID NO: 7 - DNA, 26 bp, Artificial
  synthetic
aaaacgcgtaaagctagaatcatctc

SEQ ID NO: 8 - DNA, 23 bp, Artificial
  synthetic
aatacgcgtcgacagaatgttcc

SEQ ID NO: 9 - DNA, 19 bp, Artificial
  synthetic
caagccccccaaggtgaat

SEQ ID NO: 10 - DNA, 21 bp, Artificial
  synthetic
ggcgcatggtttgttgatttc

SEQ ID NO: 11 - DNA, 23 bp, Artificial
  synthetic
atcacgcgtccttattatcttcc

SEQ ID NO: 12 - DNA, 22 bp, Artificial
  synthetic
atcacgcgtaatgttttggttc

SEQ ID NO: 13 - DNA, 24 bp, Artificial
  synthetic
tttctggaaaatctattggtagga

SEQ ID NO: 14 - DNA, 19 bp, Artificial
  synthetic
caaaacgggagtggggagc

SEQ ID NO: 15 - DNA, 22 bp, Artificial
  synthetic
aaaacgcgtctactaatcgaaa

SEQ ID NO: 16 - DNA, 22 bp, Artificial
  synthetic
aaaacgcgtctaaaatgcagat

(19) United States
(12) Patent Application Publication
(10) Pub. No.: US 2007/0077554 A1
(43) Pub. Date: Apr. 5, 2007

(54) Homologous viral internal controls for use in RT-PCR assays of enteric viruses

(76) Inventors: Sandhya U. Parshionikar, Loveland, OH (US); G. Shay Fout, Cincinnati, OH (US)

Correspondence Address: BROWDY AND NEIMARK, P.L.L.C.;624 NINTH STREET, NW, SUITE 300, WASHINGTON, DC, 20001-5303, US

(21) Appl. No.: 11/239,405

(22) Filed: Sep. 30, 2005

(51) Int. Cl. C12Q 1/70; C12Q 1/68; C12P 19/34

(52) U.S. Cl. 435/5; 435/6; 435/91.2

(57) ABSTRACT

Homologous viral internal controls have been developed for use in RT-PCR assays of a number for viruses. Two of these internal controls, such as for HAV and Norwalk virus, are cloned on a single plasmid, thereby making them useful for the multiplex detection of these viruses in a single reaction. These controls avoid the problem of inhibitors for RT-PCR that may be present in sample water assayed. These internal controls can also potentially be used in the detection of viruses in other matrices such as blood or cerebrospinal fluids or in other clinical applications. The development of two internal controls on a single plasmid enables multiplexing of multiple virus detection, thereby saving time, cost and labor.

### FIELD OF THE INVENTION

The present invention relates to internal controls for use in RT-PCR assays to detect viruses by competitive amplification, thereby making it possible to avoid false negatives.

### BACKGROUND OF THE INVENTION

Contamination of water sources by enteric viruses is a common occurrence and therefore a public health concern. There have been several reports worldwide of waterborne and foodborne outbreaks caused by noroviruses (Kukkula et al., 1997, 1999; Anderson et al., 2003; Parshionikar et al., 2003), rotaviruses (Kukkula et al., 1997; Hopkins et al., 1984; Hung et al., 1984), hepatitis A virus (Whatley et al., 1968; DeSerres et al., 1999) and enteroviruses (Amvros´eva et al., 2001). Norovirus and rotavirus are diarrhea causing viruses, with rotavirus being the leading cause of diarrhea in children (McIver et al., 2001; Bereciartu et al., 2002). Enteroviruses, that include poliovirus, coxsackie A, coxsackie B and echovirus, can cause a variety of illness such as encephalitis, meningitis, myocarditis, etc. Hepatitis A virus (HAV) belongs to the Hepatovirus group of the picornavirus family and is a major cause of infectious hepatitis in humans. All of these viruses are transmitted by the fecal-oral route. Cell culture detection of these viruses is time consuming and not possible for viruses that cannot be cultured, such as noroviruses. In recent years, molecular methods such as RT-PCR have been used to detect these viruses from environmental water samples (Abbaszadegan et al., 1999; Cho et al., 2000; Taylor et al., 2001; Fout et al., 2003). However, environmental water samples contain inhibitors of PCR (Kreader, 1996; Wilson, 1997). Several methods have been reported to remove these inhibitors prior to RT-PCR (Schwab et al., 1995; Ijzerman et al., 1997; Fout et al., 2003), but none of these methods have been proven to remove all of the inhibitors. The presence of inhibitors can lead to false negative results.

A typical way to monitor for false-negative RT-PCR results is to split processed samples and to run one of the split samples unseeded and one seeded with the virus for which the assay was developed (Fout et al., 2003). While this is an adequate approach, it increases reagent and labor costs, and decreases the amount of sample that can be used to test for additional viruses. Moreover, there is risk of accidental contamination of the sample.

The use of internal controls have been reported for HAV (Goswami et al, 1994; Atmar et al., 1995; Amal et al., 1999; Schwab et al., 2001), Norwalk virus (Atmar et al., 19951 Schwab et al., 1997), Rotavirus (Kim et al., 2002) and enterovirus (Martino et al., 1993; Arola et al., 1996) detection in shellfish, clinical samples, stool and sewage samples. However, the internal controls reported so far either have large deletions (Goswami et al, 1994; Atmar et al., 1995; Arola et al., 1996; Schwab et al, 1997) or have been derived from a foreign DNA source different from the virus (Kim et al., 2002). These internal controls can produce errors because of differences in the RT-PCR efficiency between viral and internal control templates of significantly different length of between viral and exogenous internal control templates. Others have reported creating internal controls with restriction enzyme sites built in them and not present in wild type templates (Becker-Ander and Hahlbrock, 1989; Gilliland et al., 1990). This requires digestion of the PCR product with restriction enzymes to distinguish between the test and control template, thus adding an additional step to the process. This design can also produce errors as a result of heteroduplex formation during restriction digestion or because of incomplete digestion.

Contaminated environmental water samples contain not only unknown quantities of virus, but also unknown quantities and types of PCR inhibitors.

There are three possible types of RT-PCR results that can be obtained when testing environmental water samples seeded with internal control RNA for the presence of viruses:
1. the internal control and virus do not amplify;
2. the internal control amplifies, but not the virus; or
3. the virus amplifies, but not the internal control.

The first type of result indicates the presence of PCR inhibitors. This false-negative result cannot be interpreted as the absence of virus in the sample. The second type of result should indicate the absence of virus in the sample. However, it may be obtained if the internal control is in excess over the viral RNA, such that viral amplification is suppressed. Using a minimum amount of internal control RNA can prevent this problem. In addition, suppression of virus by internal control RNA can be detected by using a “virus alone” control. The third type of result can occur if the virus is present in great excess over the internal control. This result is still a true positive for the presence of virus, and does not interfere with the purpose of the test, which is to determine the presence or absence of virus. Laboratories may want to test the effects of co-amplification when the virus is in excess over the internal control.

### SUMMARY OF THE INVENTION

It is an object of the present invention to overcome the aforementioned deficiencies in the prior art.

It is another object of the present invention to provide internal controls for assaying enteroviruses, rotaviruses, HAV and Norwalk virus.

It is another object of the present invention to obviate the need for restriction digestion in RT-PCR for viruses.

It is another object of the present invention to provide internal controls for two viruses on a single plasmid for simultaneous detection (also called multiplexing) of both viruses in one RT-PCR reaction.

The internal controls of the present invention are uniquely designed to be amplified with an efficiency that is very similar to their reactive viruses in that:
1. they are derived from their respective viruses;
2. they have the same primer binding sites as the virus;
3. they are very close in length and GC content to the virus amplicon from which they are derived;
4. internal control RT-PCR products can be distinguished from their viral RT-PCR products be polyacrylamide gel electrophoresis and DNA hybridization despite the small difference in length between the two;
5. they possess a MulI restriction site which, if desired, but is not required, can be used for further confirmation of results.
6. internal controls for two virus are uniquely designed to be present together on one plasmid vector. This feature is very useful for the simultaneous detection of two viruses simultaneously, thereby saving cost, time and labor.

The above features make these internal controls very useful in RT-PCR assays. These internal controls are not encapsidated like the viruses from which they are derived, because they were designed as RT-PCR assay controls and not as controls for virus RNA recovery from samples. In addition, they are smaller than their respective virus genomes, although their sizes are comparable to virus amplicons. These controls thereby lack the secondary and tertiary structure found in single stranded RNA viruses. Also, ROTAIC is single stranded RNA, while rotavirus has a double stranded RNA genome. These differences could make amplification efficiencies of the internal controls and their respective virus unequal. ROTAIC, however, can be converted into double stranded RNA by synthesizing both strands by in vitro transcription and then annealing them together. To determine whether efficiency differences could cause a sample to be mislabeled as a true negative result, an occasional positive control can be performed on different types of matrices by seeding samples with a low level of virus and internal controls.

Another use of the internal controls of the present invention is the quantitation of single stranded viruses by competitive amplification. The number of viral particles has been calculated form the dilution of virus at which the signal intensity of the virus derived amplicon is equal to that of the transcript derived amplicon (Atmar et al., 1995).

The internal viral controls can be added to stool extracts prior to viral RNA extraction for subsequent use in RT-PCR assays.

### BRIEF DESCRIPTION OF THE DRAWINGS

FIG. 1 is a schematic representation of constructing an internal control.

FIG. 2 illustrates RT-PCR of virus and viral internal control RNA in seeded Ohio River water concentrates.

FIG. 3 shows the sequence of virus probe region and the modified internal control region. FIG. 3A is the probe region of poliovirus. FIG. 3B is the probe region of POLOC. FIG. 3C is the probe region of HAV. FIG. 3D is the probe region of HAVIC. FIG. 3E is the probe region of Norwalk virus. FIG. 3F is the probe region of NORIC.

FIG. 3G is the probe region of rotavirus. FIG. 3G is the probe region of ROTAIC.

FIG. 4 shows separation of RT-PCR products of internal control RNA and virus on 8% polyacrylamide gel.

FIG. 4A, lane 1 is poliovirus; lane 2 is POLIC; lane 3 is a mixture of poliovirus and POLIC; lave 4 is HAV; lane 5 is HAVIC, lane 6 is a mixture of HAV and HAVIC. Lane 7 is a 123 bp ladder.

FIG. 4B, lane 1 is Norwalk virus; lane 2 is NORIC; lane 3 is a mixture of Norwalk virus and NORIC; lane 4 is a 123 bp ladder.

FIG. 4C, lane 1 is a 123 bp ladder; lane 2 is rotavirus; lane 3 is ROTAIC; lane 4 is mixture of rotavirus and ROTAIC.

FIG. 5 shows quantitation of HAV in seeded Ohio river water sample in the presence of a fixed amount of HAVIC 100 RT-PCR units (30 copies) of HAVIC used in all lanes.

Lane 1 is HAVIC+HAV (100 RT-PCR units). Lane 2 is HAV (100 RT-PCR units). Lane 3 is HAVIC. Lane 4 is HAVIC+HAV (50 RT-PCR units). Lane 5 is HAV (50 RT-PCR units). Lane 6 is HAVIC+HAV (20 RT-PCR units). Lane 7 is HAV (20 RT-PCR units). Lane 8 is HAVIC+HAV (10 RT-PCR units). Lane 9 is HAV (10 RT-PCR units). Lane 10 is HAVIC+HAV (5 RT-PCR units). Lane 11 is HAV (5 RT-PCR units). Lane 12 is HAVIC+HAV (1 RT-PCR unit). Lane 13 is HAV (1 RT-PCR unit). Lane 14 is RT-PCR negative control. Lane 15 is a 123 bp ladder.

FIG. 6 is a representative dot blot hybridization of RT-PCR reactions performed with water samples seeded with HAV and HAVIC, PV and POLIC, and Norwalk virus and NORIC.

FIG. 6A and 6B show sot blot hybridization performed with water samples seeded with HAV and HAVIC; (A) was probed with HAV probe, B was probed with HAVIC probe.

A1 and B1: HAVIC+HAV (1 RT-PCR unit)

A2 and B2: HAV (1 RT-PCR unit)

A3 and B3: HAVIC

A4 and B4: HAVIC+HAV (10 RT-PCR units)

A5 and B5: HAV (10 RT-PCR units)

A6 and B6: RT-PCR negative control

A7 and B7: HAV positive control

A8 and B8: HAVIC positive control C and D: dot blot hybridization of RT-PCR reactions performed with water samples seeded with PV and POLIC; C probed with enterovirus probe, D probed with POLIC probe.

C1 and D1: POLIC+PV (20 RT-PCR units)

C2 and D2: PV (20 RT-PCR units)

C3 and D3 POLIC (10 RT-PCR units)

C4 and D4: POLIC+PV (10 RT-PCR units)

C5 and D5: PV (10 RT-PCR units)

C6 and D6: RT-PCR negative control

C7 and D7: POLIC positive control

C8 and D8: PV positive control

E and F: show dot blot hybridization of RT-PCR reactions performed with water samples seeded with Norwalk virus and NORVIC. E was probed with Norwalk probe, and F was probed with NORIC.

E1 and F1: NORIC+Norwalk virus (100 RT-PCR units)

E2 and F2: Norwalk virus (100 RT-PCR units)

E3 and F3: NORIC (100 RT-PCR units)

E4 and F4: NORIC+Norwalk virus (10 RT-PCR units)

E5 and F5: Norwalk virus (10 RT-PCR units)

E6 and F6: RT-PCR negative control

E7 and F7: Norwalk virus positive control

E8 and F8: NORIC positive control.

FIG. 7 describes construction of a plasmid having both HAV internal control and Norwalk virus internal control.

### DETAILED DESCRIPTION OF THE INVENTION

Homologous viral controls for hepatitis A, poliovirus, Norwalk virus and rotavirus have been developed. These internal controls are used in RT-PCR assay for detection of these viruses by competitive amplification, thereby making it possible to detect false negatives in processed water samples. These internal controls can also be used in samples other than water, such as body fluids like blood and cerebrospinal fluid, to test for the presence of HAV and enteroviruses.

Each internal control has the same primer binding sites as its respective wild type virus. Each is developed such that it is very similar to its wild type virus amplicon in its length and GC content, both of which are known to affect the thermodynamics and efficiency of PCR (McCulloch et al., 1995). Despite the similarity in length to the viral RT-PCR product, each internal control RT-PCR product can be distinguished from the formed by polyacrylamide gel electrophoresis and DNA hybridization, thereby obviating the need for restriction digestion. Also, internal controls for two viruses, such as, HAV and Norwalk virus were designed to be present on a single one-plasmid vector so that two viruses can be detected simultaneously in a single RT-PCR reaction. The process by which internal controls were developed in this invention can be extrapolated for making internal controls for other viruses as well, particularly for making internal controls for more than one virus so that more than one virus can be detected in a single RT-PCR reaction.

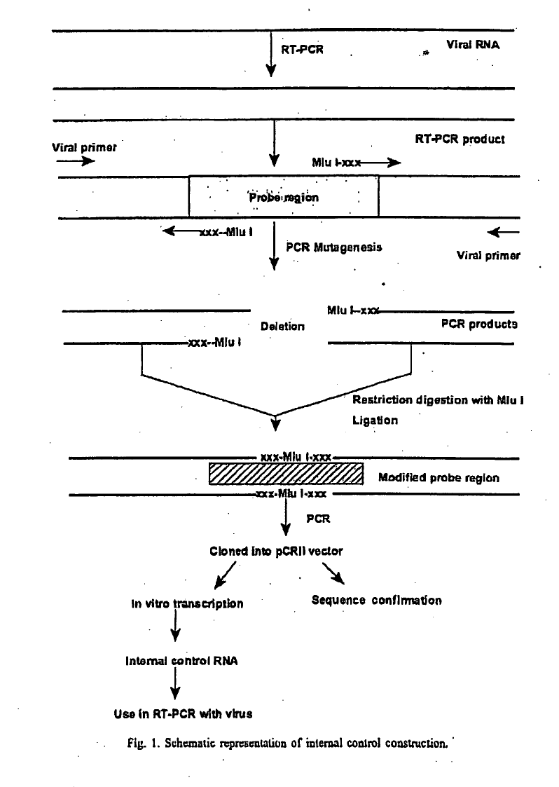

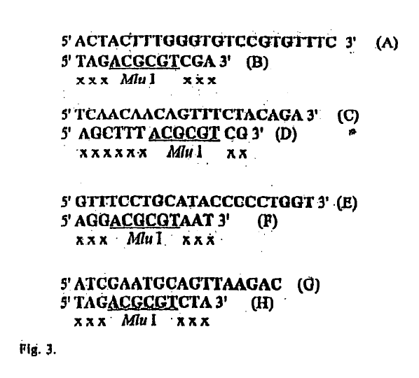

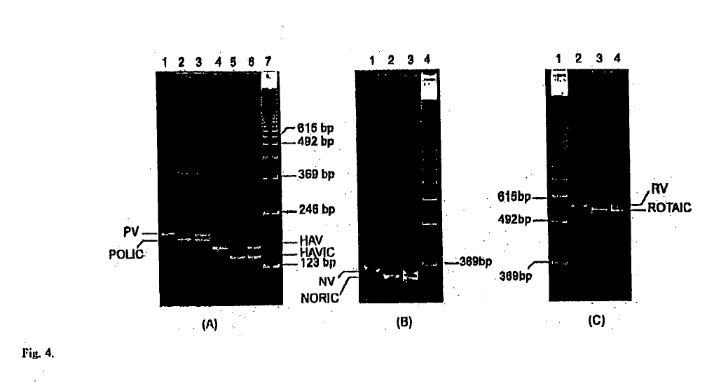

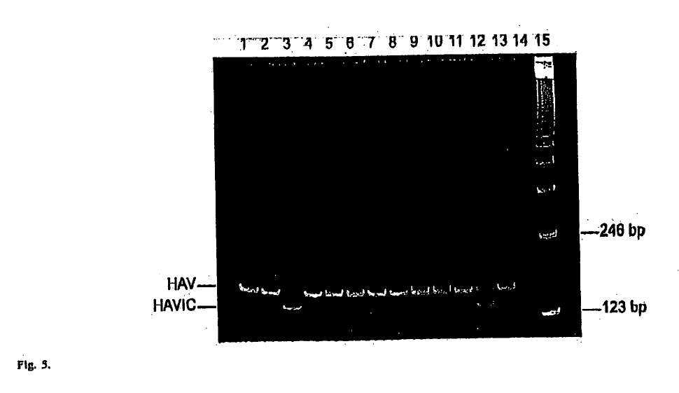

Internal controls for HAV, poliovirus, Norwalk virus and rotavirus amplification were constructed by PCR mutagenesis, as shown in FIG. 1. The accuracy of the desired sequence manipulations were confirmed by sequencing, as shown in FIG. 3. The deletions created in the probe region of each internal control were: 11 bases from POLIC, 15 basis from HAVIC, 9 bases from NORIC and 6 bases from ROTAIC. Te RE-PCR product length of HAVIC, POLIC, NORIC and ROTAIC is 146, 184, 352, and 55 bp, respectively. Viral RT-PCT products were separated from their respective internal control RT-PCR products by electrophoresis on 8% polyacrylamide gel, as shown in FIG. 4.

After developing internal controls for HAV and Norwalk virus individually, each of them was cut with restriction enzyme E.CoRI to release the inserted fragment. The HAV and Norwalk internal control fragments were then ligated together. This ligated mixture was then amplified with one HAV primer (HAV 11) and one Norwalk primer (MRD 212). These primers were chosen such that only the rightly oriented clones could amplify. The amplified product was then cloned into a plasmid with T7 and SP6 promoters for in-vitro transcription. This process is depicted in FIG. 7. DNA of internal controls for all the viruses was sequenced to confirm the accuracy of the cloned regions.

Materials and Methods

Viruses

The origin and preparation of hepatitis A virus, Norwalk virus and rotavirus stocks were previously described (Fout et al., 2003). The titers of the stocks used were: hepatitis A virus strain HM-175, 1.6×10⁸ PFU/ml (plaque forming unit); Norwalk virus, about 2×10⁶ PFU/ml. (an RT-PCR unit is defined in terms of the highest dilution of a virus stock that gives a detectable signal on an agarose gel); rotavirus, Wa strain, 2000 PFU/ml. Poliovirus, Mahoney strain, was grown in Buffalo Green Monkey kidney cells, and the virus was released by thee cycles of freeze-thaw. Cell debris was removed by centrifugation at 20,000 rpm and the virus was aliquoted and stored at −70° C. The stock virus had a titer of 8.3×10⁸ PFU/ml.

Primer Design

Sequence alignment was performed on strain isolates of each virus using MegAlign version 5.03 (DNASTAR, INC.), and primers were designed from the conserved regions using oligo 5.0 software (National Biosciences, Inc., Plymouth, Minn.). The primers designed for each virus group were identical to most virus strains in the grout, although some strains had mismatches in the central or 5′ region. Sequences of the primers used are shown in Table 1.

TABLE 1

Primers used in the study

| | | Virus | Genome |
|---|---|---|---|
| Primer | Sequence (5′-3′) | group | location |
| MRd 13 | ACCGGATGGCCAATCCAA | En- | 621-638^{a} |
| (RT | | tero- | |
| primer) | | virus | |
| MRD14 | CCTCCGGCCCCTGAATG | En- | 444-460^{a} |
| (PCR | | tero- | |
| primer) | | virus | |
| POLV1 | TCTTTAACGCGTCGACCTTTTAT | En- | 562-569^{a} |
| (muta- | | tero- | (region |
| genesis | | virus | of homol |
| primer) | | | |
| POLV2 | AAATACGCGTCTATCGGTTCC | En- | 543-550^{a} |
| (muta- | | tero- | (region |
| genesis | | virus | of homol |
| primer) | | | |
| HAV11 | GTTAGAGTGAATGTTTATCTTTCAGCA | Hepa- | 2127- |
| (PCR | | to- | 2153^{b} |
| primer) | | virus | |
| HAV12 | GGTTGTTATACCAACTTGGGGA | Hepa- | 2267- |
| (RT | | to- | 2288^{b} |
| primer) | | virus | |
| HAV1 | AAAACGCGTAAAGCTAGAATCATCTC | Hepa- | 2211- |
| (muta- | | to- | 2222^{b} |
| genesis | | virus | (region |
| primer) | | | of hom |
| HAV2 | AATACGCGTCGACAGAATGTTCC | Hepa- | 2252- |
| (muta- | | to- | 2263^{b} |
| genesis | | virus | (region |
| primer) | | | of homo |
| MRD 211 | CAAGCCCCCCAAGGTGAAT | Noro- | 5541- |
| (PCR | | virus | 5559^{c} |
| primer) | | | |
| MRD 212 | GGCGCATGGTTTGTTGATTTC | Noro- | 5881- |
| (RT | | virus | 5901^{c} |
| primer) | | | |
| NORV1 | ATCACGCGTCCTTATTATCTTCC | Noro- | 5719- |
| (muta- | | virus | 5729^{c} |
| genesis | | | (region |
| primer) | | | of hom |
| NORV2 | ATCACGCGTAATGTTTTGGTTC | Noro- | 5749- |
| (muta- | | virus | 5758^{c} |
| genesis | | | (region |
| primer) | | | of hom |
| ROTA11 | TTTCTGGAAAATCTATTGGTAGGA | Rota- | 107-130^{d} |
| (PCR | | virus | |
| primer) | | | |
| MRD155 | CAAAACGGGAGTGGGGAGC | Rota- | 1222- |
| (RT | | virus | 1240^{d} |
| primer) | | | |
| ROTAV1 | AAAACGCGTCTACTAATCGAAA | Rota- | 242-251^{d} |
| (muta- | | virus | (region |
| genesis | | | of homol |
| primer) | | | |
| ROTAV2 | AAAACGCGTCTAAAATGCAGAT | Rota- | 271-279^{d} |
| (muta- | | virus | (region |
| genesis | | | of homol |
| primer) | | | |

All primers were designed using tho Oligo 5.0 software (National Biosciences Inc., Plymouth, MN).
^{a}Accession number NC002058.
^{b}Accession number M14707.
^{c}Accession number NC001959.
^{d}Accession number M33608.

PCR Mutagenesis for Internal Controls Construction

RT-PCR products were generated from each stock virus using the group-specific primers from Table 1 and the RT-PCR conditions described below. The probe region of each viral RT-PCR product to which probes were hybridized in dot blot hybridization assays was modified by PCR mutagenesis to create the internal controls. Two primers, one at each end of the probe region of each viral RT-PCR product, were designed such that an MluI site was attached to the 5′ end followed by two to six base mutations and a stretch of 8-12 base homology with the respective viral genome at its 3′1, as shown in FIG. 1.

One of these primers was used with the RT primer and the other with the PCR primer specific for the virus. Using these two sets of primers, two PCRs were performed with the RT-PCR product of the respective viruses as the template. The products of each PCR were cut with MluI (New England Biolabs, Beverly, Mass.) and then ligated to each other overnight with T4DNA ligase (New England Biolabs) at 14° C. This ligated product was amplified for 25 cycles with RT and PCR primers specific for each virus under PCR conditions described below. The PCR product was then cloned in the TA cloning vector PCRII (Invitrogen, Carlsbad, Calif.) according to the manufacturer's protocol and transformed in TOP1OF5′ cells.

Screening Clones

Five liters (approximately 500 ng) of plasmid DNA obtained from several white colonies of transformed cells was cut with 1 unit of MluI at 37° C. for two hours in the presence of NEB buffer (100 mM NaCl, 50 mM Tris-HCl, 10 mM MgCl₂, 1 mM dithiothreitol). Since MluI site is not present in the amplified region of viral RNA or in the vector, the clones that appeared linearized with MluI on a 1% agarose gel (Ameresco, Solon, Ohio) when stained with ethidium bromide were taken as putative internal controls. The plasmids containing the internal control regions were named as follows: pHAVIC (hepatitis A internal control), pPOLIC (poliovirus internal control), pNORIC (Norwalk virus internal control) and PROTAIC (rotavirus internal control).

Sequencing

To confirm the accuracy of the manipulated region, the probe region of each internal control was sequenced in both directions with T7 and SP6 primers using the ABI Prism Big Dye Terminator Cycle Sequencing Ready Reaction kit. on an ABI Prism 3700 DNA analyzer.

In-vitro Transcription of Internal Control DNA

PHAVIC, PPOLIC, PNORIC and pROTAIC were linearized with HindIII and BamH (new England Biolabs). These enzymes were chosen because the inserts in the plasmids did not have restriction sites for them. One microgram of these linearized templates was used for making RNA with MAXIscript in vitro transcription kit (Ambicon, Austin Tex.) according to the manufacturer's protocol.

DNA from the transcription product was removed by addition of 20 U of RNAse free DNAse I (Ambion Inc., Austin, Tex.) and incubation for 30 minutes at 37° C. The reaction was treated once with acid phenol:chloroform:isoamyl alcohol, pH 4.5 (Ambion Inc.) and precipitated with 5 M ammonium acetate (Ambion Inc.) and chilled ethanol (Aaper Alcohol and Chemical Co., Shelbyville, Ky.). The pellet was resuspended in 18 microL of DEPC treated water and 2 μL of the 10× transcription buffer provided with the kit. The RNA preparation was once again treated with 20 U of DNAse I for 30 minutes at 37° C. followed by acid phenol extraction and ethanol precipitation. The pellet was suspended in 50 μl of RNA storage buffer (1 mM sodium citrate, pH 6.5, Ambion Inc.). Since unincorporated nucleotides and transcription products of varying sizes can contribute to the absorbance, they were removed by gel elution of the appropriate RNA band from a denaturing acrylamide gel. Specifically, 25 μl or RNA was loaded along with an RNA marker, each on two 5% acrylamide-8 M urea denaturing gels. The gels were run at 150 V for one hour. One gel was stained with ethidium bromide for visualizing the bands. The other gel was used for UV shadowing wherein the gel was placed over a piece of SARAN WRAP and then placed onto a fluor coated TLC plate (Ambion, inc.) Short wave UV light was directed onto the gel with a hand held UV lamp. The full-length transcript was excised with a sterile scalpel and placed into 350 μl of probe elution buffer (0.5 M ammonium acetate, 1 mM EDTA, 0.2% SDS). The tube was incubated at 37° C. overnight, after which it was centrifuged at 15,000 rpm for five minutes to remove pieces of gel. RNA from the supernatant was precipitated by ethanol precipitation. The RNA pellet was suspended in RNA storage buffer (Ambion Inc.) and O.D. at A₂₆₀ was measured on a UV spectrophotometer. The concentration of each internal control RNA was as follows: HAVIC, 9.6 μg/ml; POLIC, 4 μg/ml; NORIC, 18.56 μg/ml; ROTAIC, 26 μg/ml.

Determining DNA Removal from Internal Control RNA Transcripts

In order to determine that contaminating plasmid DNA was removed from the in vitro transcription product (RNA) of each internal control, RT-PCR and PCR amplifications were performed on 10 fold serial dilutions of each internal control with its virus specific primers under conditions described below.

Ohio River Water Sample Filtration and Concentration

A fifty-gallon (approximately 200 L) sample of Ohio River water was obtained from the pilot plant water tank of the US EPA, Cincinnati. The pH of the water was dropped to approximately 6.8 with 3 ml of 6N HCl1. The water was filtered through an IMDS electropositive cartridge filter using a peristaltic pump. The collected water sample was then eluted twice with 1.5% Adam Beef Extract, pH 9.5 (Adams Scientific, West Warwick, R.I.) and then reconcentrated using the eluate procedure (Dahling, 2002). Briefly, viruses in each eluate were concentrated onto celite at pH 3.5. The celite was collected on a sterile pre-filter (Millipore, Bedford, Mass.) and viruses were eluted with 0.15 M sodium phosphate, dibasic, pH 9.0. The pH was adjusted to 7.3 and the sample was filter sterilized by passing it through a 0.2 μM acrodisc filter (Pall Gelman Laboratory, Ann Arbor, Mich.) and frozen at −70° C.

Sample concentration and Inhibitor Removal Prior to RT-PCR

The concentrates from the first and second eluates were treated for inhibitor removal according to the method of Fout et al., 2003. Briefly, 32 ml of each concentrate was subjected to ultracentrifugation through a 30% sucrose layer for 4.5 hours and the pellet was resuspended in phosphate buffered saline (PBS) with 0.2% BSA. The resuspended pellet was then treated with an equal volume of 0.01% dithiozone, 0.01 M 8-hydroxyquinoline/butanol/methanol/trichloroethane (0.1/0.9/1/0.25/0.25, v/v) prepared with stock solutions of 0.01% dithiozone (diphenyl thiocarbazone, Fisher Scientific, Pittsburgh, Pa.) and 0.01 M 8-hydroxyquinoline (fisher Scientific) in chloroform. The samples were mixed by vortexing and the aqueous and organic phases were separated by centrifugation. The aqueous and organic phases were separated by centrifugation. The aqueous layer from each sample was removed and further concentrated on Microcon-100 filter units (Amicon Inc.) Five μl of this concentrate was used for each RT-PCR assay.

RT-PCR of Virus and Virus Internal Control RNA in Seeded Ohio River Concentrates

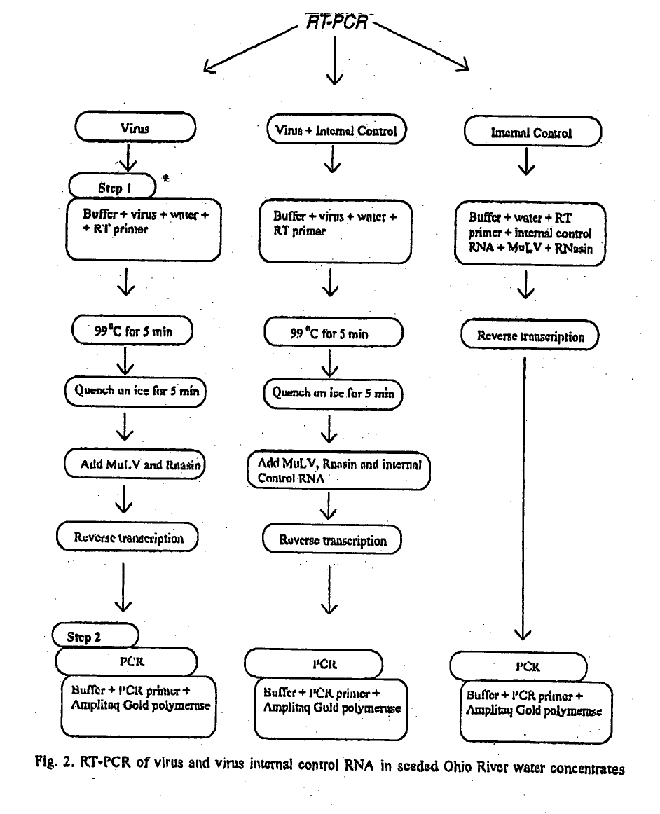

To test the degree of competition between internal control RNA and natural template, 100 RT-PCR units of HAVIC and NORIC RNA each and 10 RT-PCR units of POLIC were co-amplified with serial dilutions (from 100 to 1 RT-PCR units) of HAV, Norwalk virus, and poliovirus added to Ohio River concentrates, respectively. For each virus dilution, three conditions were tested: the virus alone, the virus+internal control, and internal control along. FIG. 2 shows the results.

In the first step of the RT-PCR, reverse transcription was performed in 30 μl volume. For virus alone, this included 5 μl of virus dilution, 5 μl of water concentration and buffer containing 10 mM Tris, pH 8.3, 50 mM KCl, 1.5. mM MgCl₂, 20 nM of each dNTP and 50 pmole of virus specific TR primer (Table 1). The reaction was overlaid with 50 μl of mineral oil and the viral RNA was released by heating at 99° C. After quenching on ice for five minutes, 7.5 units of MuLV and 30 units of Rnasin were added. In the case of virus+internal control, this was followed by the addition of 0.5 μl of respective internal control RNA dilution and the reverse transcription reaction was performed at 43° C. for 60 minutes, followed by 94° C. for five minutes. For internal control alone, the RT mix consisted of 5 μl water concentrate, buffer containing 10 mM Tris, pH 8.3, 50 mM KCl, 1.5 mM MgCl₂, 20 nM of each dNTP, 50 pmole of virus specific RT primer and 0.5 μl of respective internal control RNA dilution. The reverse transcription reaction was then performed an all three conditions at 43° C. for 60 minutes, followed by 94° C. for five minutes. The internal control RNA in both the virus+internal control and internal control alone was not subjected to 99° C. for five minutes, as unprotected RNA may be degraded at high temperatures in the presence of divalent cations (Amnion Instruction manual). PCR was performed on all three conditions by adding 70 μl of a mixture containing 10 mM Tris, pH 8.3, 50 mM KCl, 3 mM MgCl₂, 50 pmole of virus specific PCR primer and 5 units of Amplitaq Gold polymerase (applied Biosystems). The cDNA was amplified for 40 cycles, each consisting of one minute at 95° C. for one minute and 72° C. for 1.5 minute. A final incubation was carried out at 72° C. for fifteen minutes.

Polyacrylamide Gel Electrophoresis

12.5 μof the RT-PCR product from river water seeded with virus and its internal control was loaded onto an 8% polyacrylamide (acrylamide:bis-acrylamide 29:1, Sigma Chemical Company, St. Louis, Mo.) gel and run at 150 V for six to nine hours in 1XTBE in a vertical electrophoresis apparatus (Biorad Laboratories, Richmond, Calif.).

Dot Blot Hybridization

15.75 μl of each PCR product was denatured with 0.1 M NaOH and 0.4 M EDTA for ten minutes. Ammonium acetate was added at 2 M final concentration for neutralizing, and 50 μl of this mixture was spotted onto Magnagraph nylon membranes using Microsample Filtration Manifold (Schleicher & Schuell, Keene, N.H.). DNA on the membranes was UV crosslinked for one minute. The membranes were prehybridized for one hour at 51° C. and then hybridized for eighteen hours at 51° C. with individual digoxigenin-ddUTP labeled probes made with DIG Oligo 3′-end labeling kit (Roche Molecular Biochemicals, Indianapolis, Ind.). The membranes were washed at 51° C. with different salt concentrations, depending upon the probe used (see Table 2). Following the stringency wash, the membranes were blocked and treated with anti-digoxigenin-alkaline phosphatase conjugate. Hybridized probes were detected using the chemiluminescent substrate, CSPD (Trropix, Foster City, Calif.) and the blot exposed to X-ray film (Eastman Kodak, Rochester, N.Y.).

Results

Determination of Removal of DNA from Internal Control RNA

RT-PCR and PCR performed on 10-fold serial dilutions of each internal control RNA showed that while complete DNA removal was not achieved, there was a significant difference in the detection limit between internal control RNA and contaminating DNA. Specifically, the RT-PCR and PCR end point of POLIC was 10⁻⁹ and 10⁻², respectively. The RT and PCT end point of HAVIC was 10⁻¹¹ and 10⁻³, respectively. NORIC had RT %-PCR and PCR end points of 10⁻¹⁰ and 10⁻¹ respectively, while ROTAIC had RT-PCR and PCR end points of 10−7 and 10⁻³ (data not shown). These differences in detection limits were sufficient, as the dilutions (10⁻⁸, 10⁻⁹, 10⁻⁸ and 10⁻⁵ of POLIC, HAVIC, NORIC and ROTAIC, respectively) of internal control RNA that were used for co-amplification with virus had no detectable DNA contamination.

Determination of Detection Limit of Virus and its Internal Control

In order to determine the dilution of the virus and its internal control RNA to be used for co-amplification studies, the detection limit of each was determined by performing RT-PCR on 10-fold serial dilutions of the virus and its internal control RNA separately. These dilutions were performed on the original samples of stock virus and internal controls.

Five microliters of each reaction was loaded onto a 3% agarose gel and stained with ethidium bromide. The detection limits were 10⁻⁴ for HAV, 10⁻⁵ for poliovirus, 10⁻⁴ for Norwalk virus and 10⁻⁴ for rotavirus (data not shown). The detection limits for internal control RNA were 10⁻¹¹ for HAVIC, 10⁻⁹ for POLIC, 10⁻¹⁰ for NORIC and 10⁻⁷ for ROTAIC (data not shown).

Application of HAVIC, POLIC and NORIC for Assessing Inhibitor Removal from Environmental Water Samples

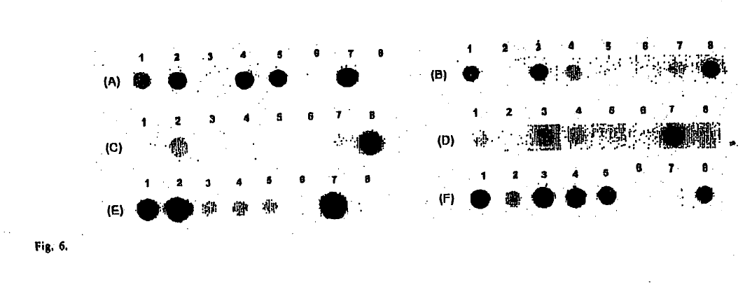

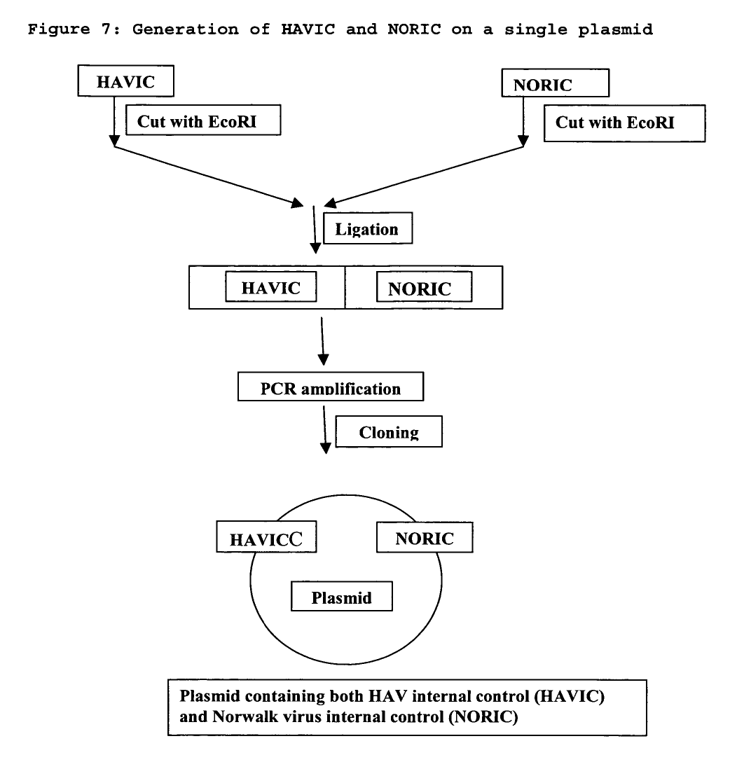

HAVIC, POLIC and NORIC DNA were used in RT-PCR assays of Ohio River water concentrates seeded with HAV, poliovirus and Norwalk virus, respectively. FIG. 5 depicts a representative polyacrylamide gel of a co-amplification assay of Ohio River water concentrate seeded with HAV and HAVIC. In all cases, internal control transcript was amplified, as seen on dot blot hybridization shown in FIG. 6, indicating satisfactory removal of PCR inhibitors. It is noteworthy that in the co-amplification reaction of each virus with its respective internal control, the virus amplicon had a detection limit similar to the virus amplicon in the virus only control. This indicated that the concentration of the internal control RNA used for co-amplification was appropriate in that it did not cause excessive suppression of virus amplification.

For example, HAV is detectable at 1 RT-PCR unit both in the co-amplification reaction and in the virus only control (FIG. 5, lanes 12 and 13 and FIG. 6, A1 and A2). Poliovirus is detectable at 20 RT-PCR units both in the co-amplification reaction and in the virus only control, but is not detectable at 10 RT-PCR units in either (FIG. 6, C1 C2, C4 and C5). Similarly, Norwalk virus is detectable at 10 RT-PCR units in the co-amplification reaction and virus only control (FIG. 6, E4 and E5). There appears to be some cross reactivity between MRD214 and NORIC probe. A southern hybridization may be useful to perform in this case. These results indicate that approximately 50-200 copies of HAVIC, 500-1000 copies of POLIC and 1000-5000 copies of NORIC are needed to obtain good co-amplification

Discussion

The internal controls of the present invention were uniquely designed to be amplified with an efficiency that is very similar to their respective viruses in that:
1.they are derived from their respective viruses;
2.they have the same primer binding sites as the virus;
3. they are very close in length and GC content to the virus amplicon from which they are derived;
4. internal control RT-PCR products can be distinguished from their viral RT-PCR products by polyacrylamide gel electrophoresis and DNA hybridization in spite of the small difference in length between the two; and
5.they possess a MulI restriction site which if desired, but not required, can be used for further confirmation of results.

The above features make these internal controls very useful in RT-PCR assays. The internal controls are not encapsidated like the viruses from which they are derived, because they were designed as RT-PCR controls and not as controls for virus RNA recovery from environmental water samples. In addition, they are smaller than their respective virus genomes, although their sizes are comparable to virus amplicons. The internal controls thereby lack the secondary and tertiary structures found in single stranded RNA viruses. Also, ROTAIC is single stranded RNA, while rotavirus has a double stranded RNA genome. These differences could make the amplification efficiencies of the internal controls and their respective virus unequal. ROTAIC, however, can be converted into double stranded RNA by synthesizing both strands by in vitro transcription followed by annealing them together. To determine whether efficiency differences could cause a sample to be mislabeled as a true negative result, an occasional positive control could be performed on different types of matrices by seeding samples with a low level of virus and internal control.

Another use for internal controls of the present invention is the quantitation of single stranded viruses by competitive amplification. The number of viral particles has been calculated form the dilution of virus at which the signal intensity of the virus derived amplicon is equal to that of the transcript deriver amplicon (Atmar et al., 1995). In this study, 1 RT PCR unit of HAV had signal intensity equal to that of 100 RT-PCR units of HAVIC. This corresponds to 100 copies (derived from calculations using the spectrophotometric readings) or HAVID (FIG. 5, line 12; FIG. 6, A1 and B1). Similarly, 20 RT-PCR units of PV had signal intensity equal to that of 10 RT-PCR units of POLIC. This corresponds to 760 copies of POLIC. These results were confirmed by dot blot hybridization, shown in FIG. 6, E1 and F1, which corresponds 4100 copies of NORIC. The internal controls of the present invention can also be added to stool extracts before viral RNA extraction of subsequent use in RT-PCTR assays.

It is to be understood that the phraseology or terminology employed herein is for the purpose of description and not of limitation. The means and materials for carrying out various disclosed functions may take a variety of alternative forms without departing from the invention.

Thus, the expressions “means to. . .” and “means for. . .” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

What is claimed is:

1. Homologous internal viral controls for assay of more than one virus in one RT-PCR reaction.

2. The internal viral controls according to claim 1 wherein the controls are for viruses selected from the group consisting of Hepatitis A virus, poliovirus, Norwalk virus, and rotavirus.

3. A method for preparing internal controls for at least a first virus and a second virus comprising:
a. developing internal controls for the first virus and the second virus individually;
b. cutting each control with restriction enzyme to release an inserted fragment;
c. ligating together the internal control fragments from the first virus and the second virus;
d. amplifying the ligated fragments with a primer for the first virus and a primer for the second virus;
e. cloning the amplified product into plasmid with promoters for in vitro transcription.

4. A PRT-PCR assay for detection of multiple viruses in a sample comprising:
a. creating a ligated fragment of viral controls from at least a first virus and a second virus;
b. amplifying the ligated fragment with a primer from the first virus and the second virus;
c. cloning the amplified product into a plasmid with promoters for in vitro transcription.

5. The RT-PCR assay according to claim 4 wherein the viruses are selected from the group consisting of Hepatitis A virus, Norwalk virus, poliovirus, and rotavirus.

6. The RT-PCR assay according to claim 4 wherein the sample is selected from the group consisting of water, blood, and cerebrospinal fluid.
